(19) United States
(12) Patent Application Publication
(10) Pub. No.: US 2023/0175011 A1
(43) Pub. Date: Jun. 8, 2023

(54) MAINTAINING DNA FRAGMENTS IN EUKARYOTIC CELLS, APPROACHES AND USES

(71) Applicant: Greffex, Inc., Houston, TX (US)

(72) Inventors: Uwe D. STAERZ, Denver, CO (US); Yan QI, Lone Tree, CO (US); Janae Wheeler CULL, Brighton, CO (US)

(21) Appl. No.: 17/907,571

(22) PCT Filed: Mar. 29, 2021
(86) PCT No.: PCT/US2021/024567

§ 371 Date: Sep. 28, 2022

Related U.S. Application Data
(60) Provisional application No. 63/001,274, filed on Mar. 28, 2020.

(51) Int. Cl. C12N 15/86

(52) U.S. Cl. CPC C12N 15/86; C12N 2710/10343

(57) ABSTRACT

Introduction of DNA fragments into eukaryotic cells exposes them to cellular enzymes, such DNases that have the ability to destroy these DNA fragments and thus reduce the function. The invention provides means and methods reducing the enzymatic destruction of linear DNA fragments transfected into cells. To this purpose, expression constructs are designed that carry genes for proteins that bind to DNA fragments and prevent the enzymatic destruction of the linear DNA fragments. The use of these genes and expression vectors in modifying packaging cells for the enhanced production of viral gene therapy vectors and methods of making these packaging cells are provided.

### FIELD

The invention relates to the field of biotechnology and molecular and cell biology; more particular, to the protection of DNA constructs introduced into eukaryotic cells from the activity of cellular DNases; to the modification of eukaryotic cells for the enhanced production of viral vectors; and more specifically to the modification of packaging cells and uses thereof for the generation of batches of recombinant viral vectors.

### BACKGROUND

Among the most commonly used vectors for the delivery of genetic material into human cells are the Adenoviruses (Ad).

Adenoviruses have been isolated from a large number of different species, and more than 100 different serotypes have been reported. The overall organization of the adenoviral genome is conserved among serotypes, such that specific functions are similarly positioned. The adenovirus genome is a linear, non-segmented, double stranded DNA, approximately 34 to 43 kilobase pairs (kbp) (size varies from group to group). The adenovirus genome is flanked on both sides by (left and right) inverted terminal repeat sequences (LITR and RITR), which are essential to replication of adenoviruses. The virus infectious cycle is divided into an early and a late phase. In the early phase, the virus is uncoated and the genome transported to the nucleus, after which the early gene regions E1-E4 become transcriptionally active.

The early region-1 (E1) contains two transcription regions named E1A and E1B. The E1A region (sometimes referred to as immediate early region) encodes two major proteins that are involved in modification of the host-cell cycle and activation of the other viral transcription regions. The E1B region encodes two major proteins, 19 K and 55 K that prevent, via different routes, the induction of apoptosis resulting from the activity of the E1A proteins. In addition, the E1B-55K protein is required in the late phase for selective viral mRNA transport and inhibition of host protein expression. Early region-2 (E2) is also divided into an E2A and E2B region that together encode three proteins, DNA binding protein, viral DNA polymerase (Pol) and pre-terminal protein (Tp), all involved in replication of the viral genome. The E3 region is not necessary for replication in vitro but encodes several proteins that subvert the host defense mechanism towards viral infection. The E4 region encodes at least six proteins involved in several distinct functions related to viral mRNA splicing and transport, host-cell mRNA transport, viral and cellular transcription and transformation.

The late proteins necessary for formation of the viral capsids and packaging of viral genomes, are all generated from the major late transcription unit (MLTU) that becomes fully active after the onset of viral DNA replication. A complex process of differential splicing and polyadenylation gives rise to more than 15 mRNA species that share a tripartite leader sequence. The early proteins E1B-55K and E4-0rf3 and Orf6 play a pivotal role in the regulation of late viral mRNA processing and transport from the nucleus.

Packaging of newly formed viral genomes in preformed capsids is mediated by at least two adenoviral proteins, the late protein 52/55 K and an intermediate protein

IVa2, through interaction with the viral packaging signal (Ψ) located at the left end of the adenoviral 5 genome. A second intermediate protein, piX, is part of the capsid and is known to stabilize the hexon-hexon interactions. In addition, piX has been described to transactivate TATA-containing promoters like the E1A promoter and the major late promoter (MLP).

### Adenovirus-Based Vectors and Adenoviral Packaging Cell Lines

Adenovirus-based vectors have been used as a means to achieve high-level gene transfer into various cell types, as vaccine delivery vehicles, for gene transfer into allogeneic tissue transplants for gene therapy, and to express recombinant proteins in cell lines and tissues that are otherwise difficult to transfect with high efficiency. The current known systems for packaging adenovirus-based vectors consist of a host cell and a source of the Adenoviral late genes.

The current known host cell lines, including the human embryonic kidney 293 (HEK293), QBI, and PERC 6 cells, express only early (non-structural) adenovirus (Ad) genes, not the adenovirus late (structural) genes needed for packaging. The adenovirus late genes have previously been provided either by the adenovirus vectors themselves, by a helper adenovirus virus, or by an expression plasmid.

“Gutless” adenoviral vectors-vectors that are devoid of all viral-protein-coding DNA sequences-have been developed. The gutless adenoviral vectors contain only the ends of the viral genome (LITR and RITR), therapeutic gene sequences, and the normal packaging recognition signal (Ψ), which allows this genome to be selectively packaged and released from cells.

### Helper Virus Dependent Adenovirus Vectors

To propagate the gutless adenoviral vector requires a helper adenovirus (the helper) that contains the adenoviral genes packaging recognition signal (Ψ). While this helper-dependent system allows the introduction of up to about 32 kb of foreign DNA, the helper virus contaminates the preparations of gutless adenoviral vectors. This contaminating replication competent helper virus poses serious problems for gene therapy, vaccine, and transplant applications both because of the replication competent virus and because of the host’s immune response to the adenoviral genes in the helper virus. One approach to decrease helper contamination in this helper-dependent vector system, has been to introduce a conditional gene defect in the packaging recognition signal (Ψ) making it less likely that its DNA is packaged into a virion. Gutless adenoviral vectors produced in such systems still have significant contaminationa with helper virus. Being able to produce gutless adenoviral gene transfer vectors without helper virus contamination offers further reduced toxicity and prolonged gene expression in animals.

### Helper Virus Independent Adenovirus Vectors

In a more recent approach to the production of gutless adenoviral vectors the adenoviral genes required for replication and packaging of an adenovirus-derived vector genome are provided by a replication-deficient circular expression plasmid that is deleted of at least of the packaging recognition signal (Ψ) and at one of the ITRs. This fully deleted helper-virus independent adenovirus (fdAd) vector platform consists of two genetic constructs, the fdAd vector genome module and the fdAd packaging expression plasmid, and a packaging cell.

The fdAd vector modules are designed to accommodate transgene constructs of up to 33 kb. They carry the left and right ITRs and packaging signals (Ψ) together with a chosen transgene, transgenes or transgene construct and, if necessary to provide a vector genome size for optimal packaging, a size compensating stuffer sequence.

The fdAd packaging plasmids are designed to provide the necessary late and early adenoviral genes of adenoviruses of different serotypes, species or animal types. They carry the adenoviral late genes, L1, L2, L3, L4, L5, together with early genes E2 and E4 in trans and are deleted of the packaging signal (Ψ) and at least one ITR.

### Packaging of Helper Virus Independent Adenovirus Vectors

fdAd vector modules are packaged into adenoviral capsids with the help of an fdAd packaging plasmid. To encapsidate an fdhiAd vector module, the fdhiAd vector genome is released from its plasmid by enzymatic cuts. A linear vector genome is created as depicted in FIG. 1. The transgene, transgenes or transgene constructs and in some instances together with inert stuffer DNA sequences are flanked by a packaging recognition signal (Ψ) and both ITRs, LITR and RITR.

The released linear fdAd vector genome module DNA together with the packaging expression plasmid DNA are transfected into a eukaryotic packaging cell (FIG. 2). The fdhiAd vector module DNA is then replicated in the packaging cells and packaged into adenovirus capsids encoded by the packaging expression plasmid. The assembled adenoviral vector carrying the fdAd vector genome can be harvested and used. The packaging expression plasmid is not being packaged.

### Packaging Cells for Helper Virus Independent Adenovirus Vectors

Packaging of fdAd vector genomes by this approach encompasses the introduction of linear DNA into packaging cells, such as HEK293-derived cells or other cells engineered to express early adenoviral genes, such as the adenoviral E1 genes. In the packaging cell the linear DNA of the fdhiAd vector genome is exposed to cellular DNases. These DNase may be cellular nucleases able to digest the free ends of linear DNA fragments. This enzymatic digestions reduces the amount of packageable of fdAd vector genomes and therefore the efficiency of vector packaging and production. In addition linear DNA fragments are toxic to mammalian cells and can lead to cell cycle arrest and/or cell death.

It has been demonstrated that certain terminal sequences on linear DNA fragments could protect linear double-stranded DNA from the activity of exonucleases in certain bacteria and eukaryotic cells. Terminal telomeric sequences of chromosomes are protected by a nucleoprotein cap that masks the ends from constitutive exposure to the DNA damage response.

Other proteins that have been found to bind to certain linear proteins. In the case of the adenovirus, the terminal protein (Tp) precursor covalently binds to a nucleotide of the ITR of the DNA chain. It remains covalently attached to the 5′-ends of the virus DNA and is cleaved to the mature Tp during virion maturation. Together with the adenovirus DNA polymerase it is responsible for the replication of the adenoviral genome. Phage Tp can also covalently attached to viral genomes priming DNA replication. In both cases the Tp prevent DNA exonucleases from enzymatically destroying linear DNA.

To protect the linear fdAd vector genomes during vector packaging, an HEK293 cell carrying the adenoviral E1 gene for transcriptional activation was modified by the stable transfection of plasmid expressing the adenoviral genes encoding the terminal protein (Tp) and DNA polymerase (Pol). For this purpose a eukaryotic expression vector was constructed that carried the adenovirus 5-derived Tp and Pol genes under the control of the birectional promoter Surfeit 1 (Surf1). The design and sequence of the vector are presented in FIG. 3.

### Adenovirus Vectors for Gene Therapy and Protein Expression

Gene delivery or gene therapy is a promising method for the treatment of acquired and inherited diseases. An ever-expanding array of genes for which abnormal expression is associated with life-threatening human diseases are being cloned and identified. The ability to express such cloned genes in humans will ultimately permit the prevention and/or cure of many important human diseases, diseases for which current therapies are either inadequate or non-existent.

Two advances have sought to overcome the problem of anti-adenoviral immunity are the use of “gutless” (fully-deleted) adenoviral vectors and the use of rare adenoviral serotypes and the pseudotyping the adenoviral hexons. While the use of “gutless” adenoviral vectors removes the L3 gene from the therapeutic vector, the propagation of these “gutless” viruses can involved the the presence of helper adenovirus that still contains L3 genes. And these helper viruses are significant contaminants in the therapeutic preparations of the “gutless” adenoviral vectors.

The use rare adenovirus serotypes or animal adenoviruses may avoid the problem of pre-existing immunity in that fraction of patients who have not been previously exposed to a given adenovirus. Still, as the adenoviral hexon proteins are highly immunogenic, a treatments with a minimally modified adenoviral gene delivery vector based on a rare serotype induces an immune reaction, including neutralizing antibodies that interferes with the subsequent use of that adenovirus vector of that human or animal serotype or species. This immune reaction may induce inflammatory responses and interfere with the therapeutic function of the vector.

### Adenoviruses as Vaccine Vectors

Adenoviruses have transitioned from tools for gene replacement therapy to bona fide vaccine delivery vehicles. They are attractive vaccine vectors as they induce both innate and adaptive immune responses in mammalian hosts. Currently, adenovirus vectors are being tested as subunit vaccine systems for numerous infectious agents ranging from malaria to HIV-1. Additionally, they are being explored as vaccines against a multitude of tumorassociated antigens. Thus far, most efforts have focused on vectors derived from adenovirus of human or simian serotypes for eventual use as vaccines for humans, while bovine, porcine, and ovine adenoviruses have been explored for veterinary use.

The dynamics of adenoviral gene expression have made the production of true adenoviral packaging cell lines difficult: expression of the adenoviral early functional transcription region (E1A) gene induces expression of the adenoviral late genes (structural, immunogenic genes), which in turn kills the cell. Accordingly, a host cell that constitutively expresses the adenoviral early genes cannot carry the “wildtype” adenoviral late cistron. Previous host cells for propagating adenoviral vectors are not “packaging” cells. Specifically, the 293, QBI and PER.C6 cells express only early (non-structural) adenoviral genes, not the adenoviral late genes needed for packaging. The adenoviral late genes have previously been provided either by the adenoviral vector itself or by a helper adenoviral virus. These adenoviral late genes in the adenoviral vector or in a helper adenoviral virus contribute to the inflammatory response to the adenoviral vector; interfere with the immune response to adenoviral based vaccines; induce immune non-responsiveness to adenovirus in allogeneic transplant applications, and contribute to contamination in adenoviral based protein expression. Further, they occupy

### SUMMARY

The present invention addresses the problem of DNA stability in eukaryotic cells and provides an engineered cell system and its uses for the protection of linear DNA fragments of DNA within eukaryotic cells and to the production of adenovirus-based gene transfer vectors.

According to aspects illustrated herein, there is provided an engineered eukaryotic packaging cell line permissive for replication of adenoviral vectors that carries the adenovirus early region 1 (E1) coding sequences and also the adenoviral Tp and Pol, both of which are either stably integrated into the cell genome or transiently expressed in the cells with the help of an eukaryotic expression vector.

According to other aspects illustrated herein, there is provided a system of eukaryotic expression vectors that drive the expression of the adenovirus Tp and/or the gene for the adenovirus Pol, which are either stably integrated into the cell genome or transiently expressed in eukaryotic cells.

According to aspects illustrated herein, there is provided a design to engineer linear DNA fragments, to which adenoviral Tp and/or Pol can bind, so that the linear DNA fragments are protected from intracellular DNases found within eukaryotic cells.

According to aspects illustrated herein, there is provided a system that includes (a) an adenovirus packaging cell engineered to express adenovirus E1, Tp and/or Pol; (b) a fdAd vector genome model; and (c) a packaging expression plasmid, wherein the fdAd vector genome and the packaging construct are transfected into the adenovirus packaging cell, resulting in the encapsidation of a fdAd vector genome independently of a helper adenovirus.

According to aspects illustrated herein, there is provided an adenovirus packaging cell engineered to express adenovirus E1, Tp and/Pol, that enhances the packaging of minimally modified, such as E1-deleted, non-fully deleted adenovirus vectors.

Other objects and features will be in part apparent and in part pointed out hereinafter.

### DESCRIPTION OF THE DRAWINGS

FIG. 1 describes the design of an fdAd vector construct together with the restriction enzyme sites including a multiple cloning site (MCS) that allow the accommodation of different genes of interests.

FIG. 2 describes the process of packaging of a fdAd vector genome (GreVac module) into an adenoviral capsid. The linearized fdAd vector genome is cotransfected with a packaging expression plasmid (pPaCh) into packaging cells (host cells) where the fdAd vector genome is replicated and packaged into adenoviral capsids encoded on the packaging expression plasmid.

FIG. 3 depicts a eukaryotic expression vector designed to drive the expression of the adenoviral terminal protein and DNA polymerase. Expression of the genes for Tp and Pol is driven from the human bidirectional Surfeit 1 promoter.

FIG. 4 depicts the transduction of eukaryotic cells with fdAd-derived adenovirus vectors that carry a green fluorescent protein (GFP). The extent of green fluorescence was detected on a fluorescence microscope. The fdAdGFP vector were encapsidated by cotransfection of the fdAdGFP vector genome and a packaging expression plasmid into unmodified HEK293 cells.

FIG. 5 compares the packaging efficiency of unmodified parental HEK293 cells with that of the cloned line of Q7 cells that had been transfected to stably express the adenoviral terminal protein and DNA polymerase. The harvested packaged fdAdGFP vectors were used to transduce eukaryotic cells. The extent of green fluorescence was detected on a fluorescence microscope.

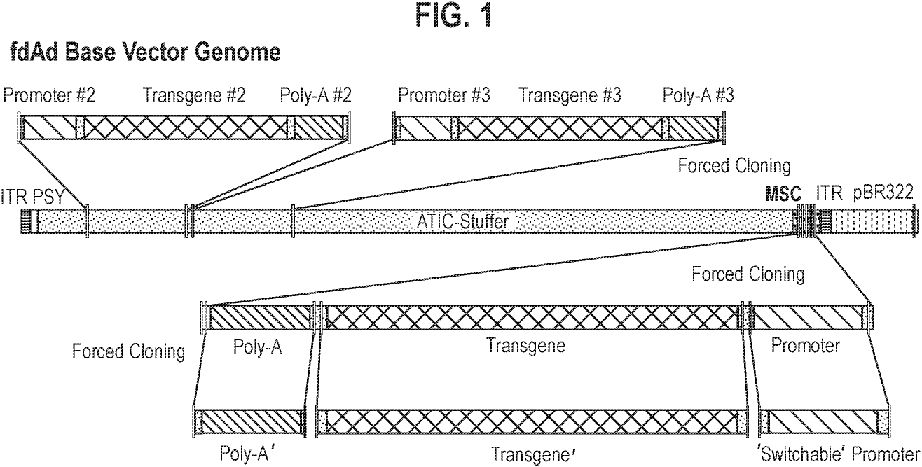

### DETAILED DESCRIPTION

The present disclosure provides, among other things an improved packaging cell for the encapsidation of adenoviral vector genomes, including but not limited to fdAd vector genomes, with or without the participation of a packaging expression plasmid.

The encapsidated adenovirus vectors by this improved packaging cell find use as gene transfer vectors for gene and protein expression, vaccine development and immunosuppressive therapy.

In an embodiment, the genes for adenovirus Tp and Pol are derived from an adenovirus of the human serotype 5. In other embodiments, the genes for the adenovirus Tp and Pol are derived from adenoviruses of other human serotypes or of simian or other animal adenovirus types.

In other embodiments, the genes for Tp and Pol are derived from other viruses.

In an embodiment, the fdAd vector genome is derived from an adenovirus of the human serotype 5. In other embodiments, the fdAd vector genome is derived from adenoviruses of other human serotypes or of simian or other animal adenovirus types.

In an embodiment, a linear DNA fragment is defined as a double-stranded DNA fragment not linked with a closed circular configuration. In another embodiment, a linear DNA fragment is defined as a single-stranded DNA fragment not linked with a closed circular configuration.

Unless defined otherwise, all technical and scientific terms used herein have the same meaning as commonly understood by one of ordinary skill in the art to which this disclosure belongs. Generally, the nomenclature used herein and the laboratory procedures in cell culture, molecular genetics, and nucleic acid chemistry and hybridization described below are those well known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer’s specifications.

The techniques and procedures are generally performed according to conventional methods in the art and various general references (see generally, Sambrook et al., Molecular Cloning: A Laboratory Manual, 2d ed. (1989) Cold Spring Harbor Laboratory Press, Cold Spring Harbor, N.Y., which is incorporated herein by reference), which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5’h edition, 1993). As employed throughout the disclosure, the following terms, unless otherwise indicated, shall be understood to have the following meanings and are more fully defined by reference to the specification as a whole.

The terms “adenovirus” and “adenoviral particle” as used herein include any and all viruses that may be categorized as an adenovirus, including any adenovirus that infects a human or an animal, including all groups, subgroups, and serotypes. Thus, as used herein, “adenovirus” and “adenovirus particle” refer to the virus itself or derivatives thereof and cover all serotypes and subtypes and both naturally occurring and recombinant forms. In one embodiment, such adenoviruses infect human cells. Such adenoviruses may be wildtype or may be modified in various ways known in the art or as disclosed herein. Such modifications include modifications to the Adenovirus genome that is packaged in the particle in order to make an infectious virus. Such modifications include deletions known in the art, such as deletions in one or more of the E1a, E1b, E2a, E2b, E3, or E4 coding regions.

An “adenovirus packaging cell” or a “packaging cell” or “host cells” as understood herein is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced.

The particles also can require certain proteins necessary for the maturation of the viral particle. Such proteins can be provided by a vector, a packaging construct or by the packaging cell. Exemplary packaging cells that may be used to make a packaging cell line according to the present invention include and cells that may be modified to express Tp and/or Pol, but are not limited to A549, HeLa, MRC5, W138, CHO cells, Vera cells, human embryonic retinal cells, or any eukaryotic cells.

Some cell lines that may be modified by the expression of adenoviral gene Tp and/or Pol, include adipocytes, chondrocytes, epithelial, fibrobasts, glioblastoma, hepatocytes, keratinocytes, leukemia, lymphoblastoid, monocytes, macrophages, myoblasts, and neurons. Other cell types include, but are not limited to, cells derived from primary cell cultures, e.g., human primary prostate cells, human embryonic retinal cells, human stem cells. Eukaryotic dipolid and aneuploid cell lines are included within the scope of the invention. As an adenovirus packaging cell the cell must be one that is capable of expressing the products of the fdAd vector genome and/or packaging expression plasmidat the appropriate level for those products in order to generate a high titer stock of recombinant gene transfer vectors.

A “coding sequence” or a sequence which “encodes” a selected polypeptide, is a nucleic acid molecule, which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and

a translation stop codon at the 3′ (carboxy) terminus A transcription termination sequence may be located 3′ to the coding sequence. Transcription and translation of coding sequences are typically regulated by “control elements,” including, but not limited to, transcription promoters, transcription enhancer elements, Shine and Delagamo sequences, transcription termination signals, polyadenylation sequences (located 3′ to the translation stop codon), sequences for optimization of initiation of translation (located 5′ to the coding sequence), and translation termination sequences.

The term “construct” refers to at least one of a fully deleted adenoviral vector construct, a packaging expression plasmid, or any other plasmid and linear DAN designed to drive expression of certain genes.

The term “delete” or “deleted” as used herein refers to expunging, erasing, or removing.

The term “E1 region” as used herein refers to a group of genes present in the adenovirus genome. These genes, such as, but not limited to, E1A and E1B, are expressed in the early phase of virus replication and activate the expression of the other viral genes. In an embodiment, an adenoviral packaging cell line of the present disclosure includes all coding sequences that make up the E1 region.

The term “terminal protein” as used herein refers to a protein whose gene is present in the genome of an adenovirus or another virus.

The term “DNA polymerase” as used herein refers to a protein whose function is the duplication of a DNA sequence. In an embodiment, the DNA polymerase is encoded by an adenovirus.

In an embodiment, an adenoviral packaging cell line of the present disclosure includes some coding sequences that make up the E1 region (for example, E1A or E1B).

In an embodiment, an adenoviral packaging cell of the present disclosure includes coding sequences that make up the terminal and/or polymerase genes.

The term “expression” refers to the transcription and/or translation of an endogenous gene, transgene or coding region in a cell.

The terms “fully-deleted adenoviral vector”, “fdAd”, “gutless”, “gutted”, “mini”, “fully-deleted”, “D”, or “pseudo” vectors as used herein refers to a linear, double-stranded DNA molecule with inverted terminal repeats (ITRs) separated by approximately 28 to 37 kb, the viral packaging signal (¥), and at least one DNA insert (all or a fragment of at least one gene of interest.

Regulation of gene expression can be accomplished by one of 1) alteration of gene structure: site-specific recombinases (e.g., Cre based on the Cre-loxP system) can activate gene expression by removing inserted sequences between the promoter and the gene; 2) changes in transcription: either by induction (covered) or by relief of inhibition; 3) changes in mRNA stability, by specific sequences incorporated in the mRNA or by siRNA; and 4) changes in translation, by sequences in the mRNA.

No viral coding genes are comprised in the fdAd. fdAds are also called “high-capacity” adenoviruses because they can accommodate up to 36 kilo bases of DNA. As vector capsids package efficiently only DNA of 75-105% of the whole adenovirus genome, and as therapeutic expression cassettes usually do not add up to 36 kb, there is a need to use “stuffer” DNA in order to complete the genome size for encapsidation.

Certain fdAd are referred to as “helper-dependent” adenoviruses because they need a helper adenovirus that carries essential adenovirus coding regions.

As used herein, the term “gene expression construct” refers to a promoter, at least a fragment of a gene of interest, and a polyadenylation signal sequence.

A “gene of interest” can be one that exerts its effect at the level of RNA or protein. Examples of genes of interest include, but are not limited to, therapeutic genes, immunomodulatory genes, virus genes, bacterial genes, protein production genes, inhibitory RNAs or proteins, and regulatory proteins. For instance, a protein encoded by a therapeutic gene can be employed in the treatment of an inherited disease, e.g., the use of a eDNA encoding the cystic fibrosis transmembrane conductance regulator in the treatment of cystic fibrosis. Moreover, the therapeutic gene can exert its effect at the level of RNA, for instance, by encoding an antisense message or ribozyme, an siRNA as is known in the art, an alternative RNA splice acceptor or donor, a protein that affects splicing or 3′ processing (e.g., polyadenylation), or a protein that affects the level of expression of another gene within the cell (i.e., where gene expression is broadly considered to include all steps from initiation of transcription through production of a processed protein), perhaps, among other things, by mediating an altered rate of mRNA accumulation, an alteration of mRNA transport, and/or a change in post-transcriptional regulation.

As used herein, the phrase “gene therapy” refers to the transfer of genetic material (e. g., DNA or RNA) of interest into a host to treat or prevent a genetic or acquired disease or condition. The genetic material of interest encodes a product (e. g., a protein polypeptide, peptide or functional RNA) whose production in vivo is desired. For example, the genetic material of interest can encode a hormone, receptor, enzyme or (poly-) peptide of therapeutic value. Examples of genetic material of interest include DNA encoding: the cystic fibrosis transmembrane regulator (CFTR), Factor VIII, low density lipoprotein receptor, betagalactosidase, alpha-galactosidase, beta-glucocerebrosidase, insulin, parathyroid hormone, and alpha-1-antitrypsin.

By “gene transfer vector” is meant a composition of a DNA construct able to transfer genetic material to cells and tissues including an encapsidated fully-deleted adenovirus- based vector of the present disclosure packaged without helper adenovirus.

The term “helper-independent” as used herein refers to the process for creating an encapsidated fully-deleted adenovirus-based gene transfer vector that does not need the presence of a helper virus for its replication.

Adenovirus vectors include “first-generation” and “second-generation” adenovirus vectors. A first-generation adenovirus vector refers to an Adenovirus in which exogenous DNA replaces the E1 region, or optionally the E3 region, or optionally both the E1 and E3 region. A second-generation adenovirus vector refers to a first-generation adenovirus vector, which, in addition to the E1 and E3 regions, contains additional deletions in the E2 region, the E4 region, or any other region of the adenovirus genome, or a combination thereof.

The term “helper virus” as used herein refers to virus used when producing copies of a helper-dependent viral vector which does not have the ability to replicate on its own. The helper virus is used to co-infect cells alongside the gutless virus and provides the necessary enzymes for replication of the genome of the gutless virus and the structural proteins necessary for the assembly of the gutless virus capsid.

By “immune response” is preferably meant an acquired immune response, such as a cellular or humoral immune response.

The term “inverted terminal repeat” (ITR) as used herein refers to DNA sequences located at the left and right termini of the Adenovirus genome. These sequences are identical to each other, but placed in opposite directions. The length of the inverted terminal repeats of Adenoviruses vary from about 50 bp to about 170 bp, depending on the type of the adenovirus or of a virus or a different family or genus. The ITRs may contain a number of different cis-acting elements required for viral growth, such as the core origin of viral DNA replication and enhancer elements for the activation of the E1 region. The ITRs may also contain sequence and structures that allow the covalent and/or non-covalent binding of viral and/cellular proteins, such as but not limited to the Tp and Pol proteins.

“In vivo gene therapy” and “in vitro gene therapy” are intended to encompass all past, present and future variations and modifications of what is commonly known and referred to by those of ordinary skill in the art as “gene therapy”, including ex vivo applications.

The term “introducing”, as used herein refers to delivery of an expression vector for stable integration or transient maintenance of gene sequences to a cell. A vector may be introduced into the cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage). As set forth above, the vector may be a plasmid, virus or other vehicle.

The term “linear DNA” as used herein refers to non-circularized DNA molecules.

The term “naturally” as used herein refers to as found in nature; wild-type; innately or inherently.

The term “nucleic acid” refers to a deoxyribonucleotide or ribonucleotide polymer in either single- or doublestranded form, and unless otherwise limited, encompasses known analogues having the essential nature of natural nucleotides in that they hybridize to single-stranded nucleic acids in a manner similar to naturally occurring nucleotides (e.g., peptide nucleic acids).

Nucleic acids are “operably linked” when placed into a functional relationship with another nucleic acid sequence. For example, a promoter or enhancer is operably linked to a coding sequence if it affects the transcription of the sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adapters or linkers are used in accordance with conventional practice.

The term “packaging expression plasmid” or “packaging expression construct” refers to an engineered plasmid construct of circular, double-stranded DNA molecules, wherein the DNA molecules include at least a subset of Adenoviral late genes (e.g., L1, L2, L3, L4, L5, E2A, and E4) under control of a promoter. The packaging expression plasmid The packaging expression plasmid does not include a packaging signa sequence (Ψ) and more than one ITR.

The packaging expression plasmid is “replication defective”-the viral genome does not comprise sufficient genetic information alone to enable independent replication to produce infectious viral particles within a cell.

Any subtype, mixture of subtypes, or chimeric adenovirus may be used as the source of DNA for generation of the fdAd vector genome and the packaging expression plasmid.

The term “packaging signal” as used herein refers to a nucleotide sequence that is present in the virus genome and is necessary for the incorporation of the virus genome inside the virus capsid during virus assembly. The packaging signal of adenovirus is naturally located at the left-end terminus, downstream from the left inverted terminal repeat. It may be denoted as “¥”.

A cell that is “permissive” supports the expression of viral genes and/or the replication of a virus.

The term “plasmid” as used herein refers to an extrachromosomal DNA molecule separate from the chromosomal DNA. In many cases, it is circular and double-stranded.

The term “poly linker” is used for a short stretch of artificially synthesized DNA, which carries a number of unique restriction sites allowing the easy insertion of any promoter or DNA segment. The term “heterologous” is used for any combination of DNA sequences that is not normally found intimately associated in nature.

The term “promoter” is intended to mean a regulatory region of DNA that facilitates the transcription of a particular gene. Promoters usually comprise a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. A “constitutive promoter” refers to a promoter that allows for continual transcription of its associated gene in many cell types. An “inducible-promoter system” refers to a system that uses a regulating agent (including small molecules such as tetracycline, peptide and steroid hormones, nerotransmitters, and environmental factors such as heat, and osmolarity) to induce or to silence a gene. Such systems are “analog” in the sense that their responses are graduated, being dependent on the concentration of the regulating agent. Also, such systems are reversible with the withdrawal of the regulating agent. Activity of these promoters is induced by the presence or absence of biotic or abiotic factors. Inducible promoters are a powerful tool in genetic engineering because the expression of genes operably linked to them can be turned on or off at certain stages of development of an organism or in a particular tissue.

The term “propagate” or “propagated” as used herein refers to reproduce, multiply, or to increase in number, amount or extent by any process.

The term “purification” as used herein refers to the process of purifying or to free from foreign, extraneous, or objectionable elements.

The term “regulatory sequence” (also called “regulatory region” or “regulatory element”) as used herein refers to a promoter, enhancer or other segment of DNA where regulatory proteins such as transcription factors bind preferentially.

They control gene expression and thus protein expression.

The term “recombinase” as used herein refers to an enzyme that catalyzes genetic recombination, such as but not limited to the Cre, Hin, Tre or FLP recombinases or the CRISPR/Cas system DNA recombinase system. A recombinase enzyme catalyzes the exchange of short pieces of DNA between two long DNA strands, particularly the exchange of homologous regions between the paired maternal and paternal chromosomes.

The term “restriction enzyme” (or “restriction endonuclease”) refers to an enzyme that cuts double-stranded DNA. The term “restriction sites” or “restriction recognition sites” refer to particular sequences of nucleotides that are recognized by restriction enzymes as sites to cut the DNA molecule. The sites are generally, but not necessarily, palindromic, (because restriction enzymes usually bind as homodimers) and a particular enzyme may cut between two nucleotides within its recognition site, or somewhere nearby.

The term “replication” or “replicating” as used herein refers to making an identical copy of an object such as, for example, but not limited to, a virus particle.

The term “replication deficient” as used herein refers to the characteristic of a virus that is unable to replicate in a natural environment. A replication deficient virus is a virus that has been deleted of one or more of the genes that are essential for its replication, such as, for example, but not limited to, the E1 genes of an adenovirus. Replication deficient viruses can be propagated in a laboratory in cell lines that express the deleted genes.

The term “stuffer” as used herein refers to a DNA sequence that is inserted into another DNA sequence in order to increase its size. For example, a stuffer fragment can be inserted inside the Adenovirus genome to increase its size to about 36 kb. Stuffer fragments usually do not code for any protein nor contain regulatory elements for gene expression, such as transcriptional enhancers or promoters.

The term “target” or “targeted” as used herein refers to a biological entity, such as, for example, but not limited to, a protein, cell, organ, or nucleic acid, whose activity can be modified by an external stimulus. Depending upon the nature of the stimulus, there may be no direct change in the target, ora conformational change in the target may be induced.

As used herein, a “target cell” can be present as a single entity, or can be part of a larger collection of cells. Sucha “larger collection of cells” may comprise, for instance, a cellculture (either mixed or pure), a tissue (e.g., epithelial or other tissue), an organ (e.g., heart, lung, liver, gallbladder, urinary bladder, eye or other organ), an organ system (e.g., circulatory system, respiratory system, gastrointestinal system, urinary system, nervous system, integumentary system or other organ system), or an organism (e.g., a bird, mammal, particularly a human, or the like). Preferably, the organs/tissues/cells being targeted are of the circulatory system (e.g., including, but not limited to heart, blood vessels, and blood), respiratory system (e.g., nose, pharynx, larynx, trachea, bronchi, bronchioles, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including, but not limited to, brain and spinal cord, and special sense organs, such as the eye) and integumentary system (e.g., skin). Even more preferably, the cells are selected from the group consisting of heart, blood vessel, lung, liver, gallbladder, urinary bladder, eye cells and stem cells. In an embodiment, the target cells are natural stem cells or precursor cells, including but not limited to oocytes or spermatocytes. In an embodiment, the traget cells are induced stem cells or induced precursor cells.

The term “transfection” as used herein refers to the introduction into a cell DNA as DNA (for example, introduction of an isolated nucleic acid molecule or a construct of the present disclosure). A packaging cell line disclosed herein is transfected with genetic constructs that lead to the expression of certain viral genes together with at least one of a adenoviral genome or a packaging expression vector. The term “transduction” as used herein refers to the introduction into a cell DNA either as DNA or by means of a gene transfer vector. A gene transfer vector can be transduced into a target cell.

The term “vector” refers to a nucleic acid used in infection of a host cell and into which can be inserted a polynucleotide. Expression vectors permit transcription of a nucleic acid inserted therein. Some common vectors include, but are not limited to, plasmids, cosmids, viruses, phages, recombinant expression cassettes, and transposons. The term “vector” may also refer to an element which aids in the transfer of a gene from one location to another.

The term “viral DNA” as used herein refers to a sequence of DNA that is found in virus particles.

The term “viral genome” as used herein refers to the totality of the DNA that is found in virus particles, and that contains all the elements necessary for virus replication. The genome is replicated and transmitted to the virus progeny at each cycle of virus replication.

The term “virions” as used herein refers to a viral particle. Each virion consists of genetic material within a protective protein capsid.

The term “wild-type” as used herein refers to the typical form of an organism, strain, gene, protein, nucleic acid, or characteristic as it occurs in nature. Wild-type refers to the most common phenotype in the natural population. The terms “wild-type” and “naturally occurring” are used interchangeably.

The following examples illustrate various aspects of the present invention. The examples should, of course, be understood to be merely illustrative of only certain embodiments of the invention and not to constitute limitations upon the scope of the invention which is defined by the claims that are appended at the end of this description.

Having described the disclosure in detail, it will be apparent that modifications and variations are possible without departing from the scope of the claims.

### EXAMPLES

The following non-limiting examples are provided to further illustrate the present disclosure.

### Example 1

### Engineering of Terminal Protein and DNA Polymerase Expression Vectors

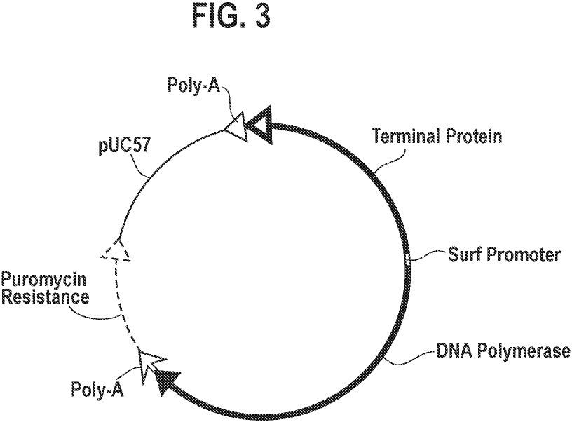

An expression vector driving the expression of the Tp and Pol genes of an adenovirus of the human serotype 5 were produced (FIG. 3 and SEQ ID NO: 1). It was engineered by moving a bi-directional expression cassette that carried both the genes for the adenoviral terminal protein (Tp) and DNA polymerase (Pol) together with the puromycin selection marker into the multiple cloning site of pUC57 (GeneScript).

The resulting Tp/Pol vector is assembled using forced cloning strategies into defined restriction enzyme recognition sites. The Tp/Pol expression vector, pUTp/Pol, is composed in the following way:
1. Vector DNA (1 - 431) and (7576 - 9860) belong to the cloning vector pUC57 (complete sequence Y 14837).
2. A DNA cassette was synthesized that was moved into the pUC57 cloning vector. It was composed of the following components:
   (i) a polyadenylation site for the Tp gene (vector DNA 440 - 505) corresponds to an “artificial” sequence;
   (ii) the adenoviral Tp gene (Vector DNA 506 - 2,521) corresponds to nucleotides 8,583 - 10,590 of the wild-type adenovirus human serotype 5 (AC 000008);
   (iii) the Surfeit bidirectional promoter (Vector DNA 2,522 - 2,689) corresponds to nucleotides 37,174 - 37,293 of the genomic sequence of human chromosome 9q34 (AC 002107);
   (iv) the DNA polymerase (Pol) gene (Vector DNA 2,690 - 6,287) corresponds to nucleotides 8,785 - 5,197 of the wild-type adenovirus human serotype 5 (AC 000008);
   (v) the a polyadenylation site for the Pol gene (Vector DNA 6,296 - 6,414) corresponds to an “artificial” sequence; and
   (vi) the puromycin selection marker (Vector DNA 6,423 - 7,575) corresponds to nucleotides 231,893 - 231,290 of the human Herpes virus 5 (AD169-BAC isolate, complete sequence AC146999).

In other embodiments, expression vectors are produced that carry the Tp and/or the Pol gene behind a constitutive or inducible eukaryotic promoter, such as but not limited to, the cytomegalovirus (CMV) immediate/early promoter, the simian vacuolating virus 40 (SV40), the elongation factor 1a (EF1a) promoter, the human ubiquitin C gene (Ubc) promoter, the human b actin promoter, or the tetracyline response element (TRE) promoter.

In other embodiments, expression vectors are produced that carry both the Tp and Pol gene separated by an internal ribosomal entry site (IRES), such as one derived from but not limited to, poliovirus, rhinovirus, hepatitis virus, or the human immunodeficiency virus.

In other embodiments, expression vectors that carry the Tp and/or Pol genes also carry positive or negative eukaryotic selection markers, such as but not limited to the following drugs, puromycin, hygromycin, G418/neomycin, or bleomycin.

In other embodiments, expression vectors that carry the Tp and/or Pol genes are used to transfect cells with the help of transfectant reagents, such as but not limited to, calciumphosphate, lipids, or polyethyleneimine.

In other embodiments, expression vectors that carry the Tp and/or Pol genes are used to transfect cells so that they are maintained in the cells transiently.

In other embodiments, expression vectors that carry the Tp and/or Pol genes are used to establish stable expression of Tp and/or Pol in the transfected cells.

### Example 2

### Engineering of Enhanced Virus Vector Packaging Cells

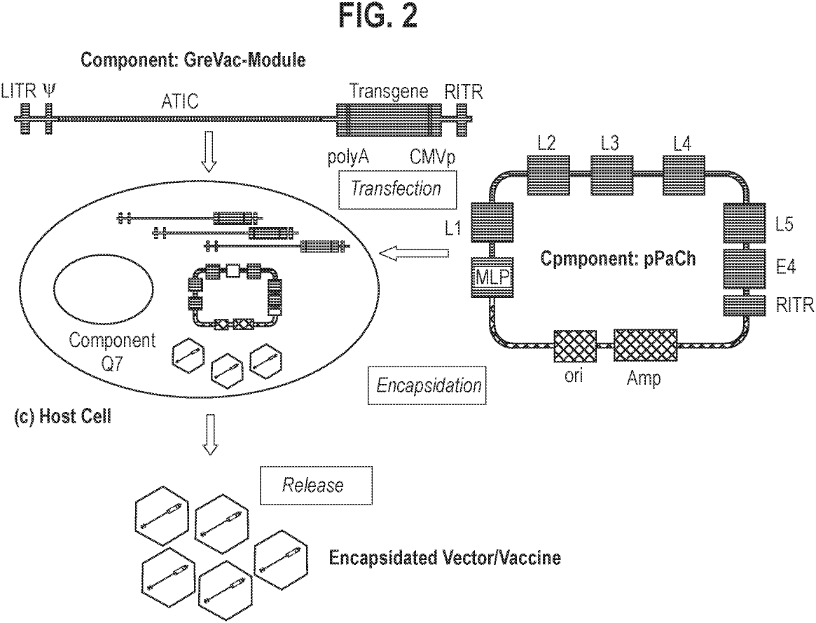

Adenoviral vectors without the use of a helper virus can be produced in the following manner as summarized in FIG. 2:
(1) Packaging cells, such as HEK293 derived cells, are transfected with a mixture of a linearized adenoviral vector genome DNA, such as but not limited to, an fdAd vector genome carrying ITRs at or close to their 5′- and 3′-ends, and a packaging expression plasmid;
(2) The transfected packaging cells are maintained in culture for some time during which the linearized adenoviral vector genome is replicated and packaged in an adenoviral capsids; and
(3) The transfected packaging cells are harvested to retrieve the encapsidated adenoviral vectors.

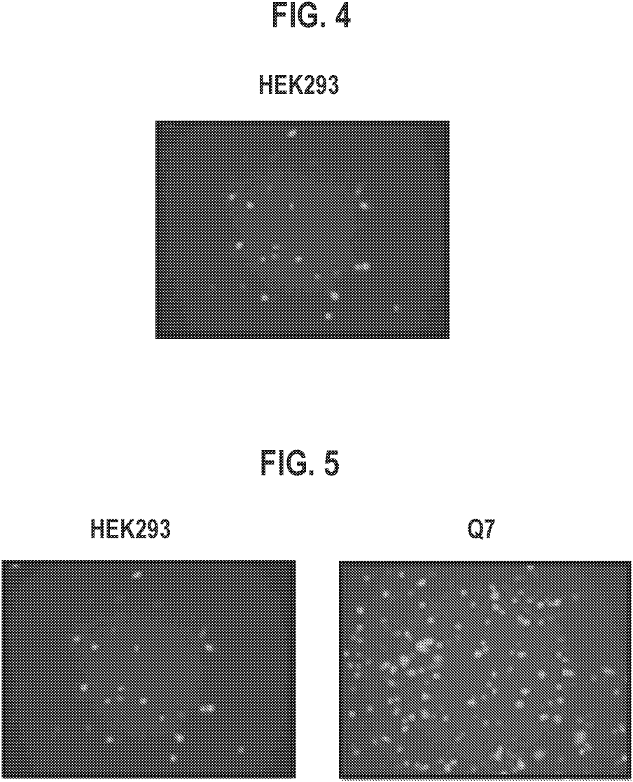

The harvested encapsidated fdAd vector was functionally tested as exemplified here with an fdAd vector carrying a green fluorescent protein behind a CMV immediate early/promoter enhancer, fdAdGFP (FIG. 4). The encapsidated fdAdGFP vector was used to transduce HEK293-derived cells. After a 24-hr culture the cells are analyzed for the expression of the GFP.

Introduction of linear viral genomes, such as but not limited to, that of adenoviral vectors, into eukaryotic cells can lead to their destruction by cellular nucleases, such as exonucleases. To enhance the active levels of linear genomes within cells, it is necessary to protect them against nucleases. A new packaging cell was engineered on the basis of HEK293 cells to enhance the rate of encapsidation of fdAd vectors. HEK293 cells had originally been immortalized by transfection with sheared adenovirus type 5 DNA (Graham, 1977). Sequencing for the adenovirus 5 insert showed that a contiguous segment of nucleotides 1 to 4,344 were integrated into chromosome 19 (19q13.2) (Louis, 1997). The HEK293 cell is known to express the adenovirus E1A and E1B genes and possibly other adenovirus genes.

The HEK293 cells were modified by a stable transfection of a eukaryotic expression vector (FIG. 3, and SEQ ID NO 1) that carries genes for the adenovirus 5 terminal protein (Tp) and DNA polymerase (Pol) under control of the bidirectional promoter Surfeit 1 (Surf 1). The Tp/Pol vector was linearized by a restriction enzyme cut with Nde1 before it was used to transfect the HEK293 cells. The transfectant cells were selected for puromycin resistance. Resistant cells were cloned. The cloned transduced packaging cells named Q7 were selected for high vector production efficiency. Stability of Tp/Pol vector integration was surveyed by measuring the expression of Tp and Pol in HTP7 cells over time by quantitative reverse transcriptase PCR (RT-PCR).

The encapsidation efficiency of the Q7 cells was compared with that of the unmodified HEK293 parent cell line. For this purpose, fdAdGFP linearized vector genomes were co-transfected with packaging expression plasmids into the two packaging cells, the Q7 cells and HEK293 parent cells. The encapsidated fdAdGFP vectors were harvested from both cell lines and used to transfect human cells. After a 24-hr culture the cells are analyzed for the expression of the GFP. As exemplified in FIG. 5, the packaging efficiency of Q7 for fdAdGFP was approximately 50-to-100-fold higher than that of the HEK293-parent cells.

### Example 3

The adenoviral terminal protein (Tp) precursor covalently binds to nucleotide within the ITR of the linear adenoviral genome. It remains covalently attached to the 5′-ends of the virus DNA and is cleaved to the mature Tp during virion maturation. Together with the adenoviral DNA polymerase (Pol) it is responsible for the replication of the adenoviral genome. The Tp by itself or in conjunction with the adenoviral Pol prevents DNA exonucleases from enzymatically destroying linear DNA.

In an embodiment, linear DNA fragments are modified by attaching adenoviral ITRs to the ends of the linear fragments. Providing the adenoviral Tp by itself or together with adenoviral Pol to eukaryotic cells that are being or have been transfected with said ITR-containing linear DNA fragments, will limit the enzymatic destruction of said ITR-containing linear DNA fragments. As a result, the said ITR-containing linear DNA fragments are maintained at higher levels in cells leading to enhanced functions of said ITR-containing linear DNA fragments.

The adenoviral Tp by itself or together with adenoviral Pol can be provided to cells by transient or stable transfection with a eukaryotic expression vectors, or as proteins or modified proteins to be introduced into cells.

### Example 4

In an embodiment, linear DNA fragments are modified at their 5′-and 3′-ends so that phage Tp can covalently attach to the linear DNA fragments. Providing the phage Tp to eukaryotic and/or prokaryotic cells that are being or have been transfected with said modified linear DNA fragments will limit the enzymatic destruction of said modified linear DNA fragments. As a result, the said modified linear DNA fragments will be maintained at higher levels in cells leading to enhanced functions of said modified linear DNA fragments.

The phage Tp can be provided to cells by transient or stable transfection with a eukaryotic expression vectors, or as a protein or a modified protein to be introduced into cells.

### Example 5

In an embodiment, linear DNA fragments are modified at their 5′-and 3′-ends so that certain proteins can attach to the linear DNA fragments. Providing such proteins, such as ones derived from but not limited to, eukaryotic and/or prokaryotic cells and able to bind to the linear DNA, to cells that are being or have been transfected with said modified linear DNA fragments, will limit the enzymatic destruction of said modified linear DNA fragments. As a result, the said modified linear DNA fragments will be maintained at higher levels in cells leading to enhanced functions of said modified linear DNA fragments.

In an embodiment, proteins can be harvested or engineered that can attach to linear and/or circular DNA fragments so that their enzymatic destruction in cells is limited. Providing such proteins, such as ones derived from but not limited to, eukaryotic cells and prokaryotic cells and able to bind DNA, to cells that are being or have been transfected with said DNA fragments will limit the enzymatic destruction of said DNA fragments. As a result, the said DNA fragments will be maintained at higher levels in cells leading to enhanced functions of said DNA fragments.

Such protective proteins can be provided to cells by transient or stable transfection with a eukaryotic expression vectors, or as a proteins or a modified protein to be introduced into cells.

### Example 6

The linear and/or circular DNA fragments that are protected from destruction within eukaryotic and/or prokaryotic by protective proteins can be used to deliver genetic constructs, such as but not limited to,
viral genomes;
genes for therapeutic purposes;
genes that code for proteins;
arrays of genes that code for proteins and/or enzymes;
genes for proteins and/or enzymes that modify cellular functions;
genes for enzymes and DNA and/or RNA constructs that modify DNA, such as but not limited to, DNA recombinases, DNA intergrases; the CRISPR machinery;
DNA and RNA fragments that are integrated into the cellular genome; and
DNA and RNA fragments that regulate cellular functions.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

What is claimed is:

1. A method of enhancing the function of a packaging cell to propagate adenoviruses and adenovirus-based gene transfer vectors comprising: (a) providing a eukaryotic expression vector that codes for the expression of a terminal protein (Tp); (b) transfecting this terminal protein expression vector into the packaging cells; and (c) introducing into the packaging cell a adenovirus derived genome that carries terminal sequences on both 5′- and 3′-ends of the linear fragments adenovirus derived genome that can be bound by the terminal protein.

2. The method of claim 1 wherein the Tp eukaryotic expression vector is transiently maintained within the packaging cell.

3. The method of claim 1 wherein the Tp eukaryotic expression vector is stably integrated into the genome of the packaging cell.

4. The method of claim 1 wherein the Tp eukaryotic expression vector codes for the expression of an adenoviral terminal protein.

5. The method of claim 1 wherein the eukaryotic expression vector that codes for the expression of an adenoviral terminal protein, also encodes for the expression of an adenoviral DNA polymerase.

6. The method of claim 1 wherein the adenovirus derived genome carries the adenoviral left and right inverted terminal repeats (ITRs) and the adenoviral packaging signal (Ψ).

7. The method of claim 1 wherein the adenovirus derived genome is partially deleted of endogenous adenoviral genes.

8. The method of claim 1 wherein the adenovirus derived genome is fully deleted of all endogenous adenoviral genes.

9. The method of claim 1 wherein the adenovirus derived genome is fully deleted of endogenous adenoviral genetic structures except the adenoviral left and right inverted terminal repeats (ITRs) and the adenoviral packaging signal (Ψ).

10. The method of claim 1 wherein the adenovirus derived genome is linear.

11. The method of claim 1 wherein the adenovirus derived genome carries modified and/or non-adenoviral genes.

12. The method of claim 1 wherein the adenovirus derived genome is used for gene medicine purposes, such as but not limited to, gene therapy, vaccination, tissue engineering and genome modifications.

13. The method of claim 1 wherein a virus or a expression vector is co-transfected into the packaging cell that provide genetic information necessary to package the adenovirus derived genome into adenoviral capsids.

14. A method of enhancing the function of DNA constructs once that have been introduced into eukaryotic cells comprising:
(a) providing a eukaryotic expression vector that codes for the expression of a protein able to protect the introduced DNA construct from enzymatic digestion;
(b) transfecting this protein expression vector into the eukaryotic cell; and
(c) introducing into the eukaryotic cell the DNA construct to which the DNA protecting protein binds.

15. The method of claim 14 wherein the eukaryotic expression vector for the protective proteins is transiently maintained within the packaging cell.

16. The method of claim 14 wherein the eukaryotic expression vector for the protective proteins is stably integrated into the genome of the packaging cell.

17. The method of claim 14 wherein the eukaryotic expression vector for the protective proteins codes for the expression of a bacteriophage or bacterial terminal protein.

18. The method of claim 14 wherein the DNA construct is circular.

19. The method of claim 14 wherein the DNA construct is linear.

20. The method of claim 14 wherein the DNA construct has been modified to enhance the binding of a bacteriophage or bacterial terminal protein.

21. The method of claim 14 wherein the DNA construct is used for gene medicine purposes, such as but not limited to, gene therapy, vaccination, tissue engineering and genome modifications.

22. A method of claim 14 wherein the eukaryotic expression vector for the protective proteins codes for proteins derived from eukaryotes or prokaryotes.

23. A method of claim 14 wherein the DNA construct has been modified to enhance the binding of said protective proteins.

24. A method of enhancing the function of DNA constructs once that have been introduced into eukaryotic cells by altering the DNA sequence of the DNA so that the enzymatic destruction of the DNA construct is reduced.

25. A method of claim 24 wherein the DNA construct is linear and wherein its 5′-and 3′-ends have been modified to reduce digestion by nucleases.

26. A method of claim 24 wherein the linear DNA constructs has been chemically altered.

27. A method of claim 24 wherein the sequence of the 5′- and 3′- ends have been modified to limit the function DNA exonucleases.


SEQUENCE LISTING (1 sequence)

SEQ ID NO: 1 - DNA, 9860 bp, Adenovirus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatttaattaatctagttcgaacacacaaaaaacacaccaacagatgtaatgaaataatataagtattattatcgatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctcagggccatcgcacccggccccgcgggcgcttccgggacgcaggaagcatctgcatccggggcgccgctgagtcccgcccagagccccgcccccggctccaggttctgcgagcggcttccgccgggctgcaggttctgcgagcggcttccgccgggctgctccgcgggcgcgtcggccatggccctggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagggccggccaataatataacatttttttcattacatctggtggtttgttttttgtgtgatgcgaactagaagcttggtgtgtgaaggccaatatgctcattggatgaggttttgtctcacacgccccaggcgcgccgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacgctagccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgcaggatcctcgagtttaaactctagaaccggtcatggccgcaataaaatatctttattttcattacatctgtgtgttggttttttgtgtgttcgaactagaagcttgctactagtatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc